(19) United States
(12) Patent Application Publication
(10) Pub. No.: US 2023/0127347 A1
(43) Pub. Date: Apr. 27, 2023

(54) ANTIBODIES

(71) Applicant: Oxford University Innovation Limited, Oxford (GB)

(72) Inventors: Rebecca POWELL DOHERTY, Oxford (GB); Nicola TERNETTE, Oxford (GB); Jack SATSANGI, Oxford (GB)

(21) Appl. No.: 17/913,457

(22) PCT Filed: Apr. 1, 2021
(86) PCT No.: PCT/GB2021/050831

§ 371 Date: Sep. 22, 2022

(30) Foreign Application Priority Data
Apr. 2, 2020 (GB) 2004916.9

(51) Int. Cl. C07K 16/24; A61P 37/00

(52) U.S. Cl. CPC C07K 16/241; A61P 37/00

(57) ABSTRACT

Provided is an anti-TNF-α antibody comprising a sequence modification that inhibits presentation of the antibody by human leukocyte antigen (HLA) molecules. The anti-TNF-α antibody may be infliximab or a fragment or variant thereof. In this case, the modification is suitably one that inhibits presentation of the antibody by DRB1*03:01, such as a modification in the region corresponding to residues 1 to 21 of SEQ ID NO: 1, or a modification in the region corresponding to residues 145 to 159 of SEQ ID NO: 1. Alternatively, the anti-TNF-α antibody may be adalimumab or a fragment or variant thereof. In this case, the modification may be one that inhibits presentation of the antibody by DQA1*05:05/DQB1*03:01. Also provided are nucleic acid sequences that encode an antibody according to the invention, and pharmaceutical composition comprising an antibody or nucleic acid sequence of the invention. The antibodies and pharmaceutical compositions of the invention may be of use in the treatment of autoimmune diseases, or disorders selected from the group consisting of: Crohn's disease; ulcerative colitis; rheumatoid arthritis; psoriatic arthritis; psoriasis; ankylosing spondylitis; and Behçet's disease.

### FIELD OF THE INVENTION

The present invention relates to modified antibodies, and to medical uses and methods of treatment employing such antibodies. It also relates to nucleic acid molecules encoding antibodies, and to pharmaceutical compositions comprising the antibodies or nucleic acid molecules. It further relates to cells comprising nucleic acid molecules of the invention, and to methods of producing antibodies using the cells of the invention.

### INTRODUCTION

Autoimmune diseases, such as inflammatory bowel diseases, are frequently not able to be cured. However, the introduction of biological therapies has dramatically improved patient outcomes. First-line biologics include anti-TNF-α monoclonal antibodies, including infliximab and adalimumab. However, the efficacy of these drugs is limited in almost 50% of patients by the development of immunogenic antibodies against the drugs.

### SUMMARY OF THE INVENTION

In a first aspect the invention provides an anti-TNF-α antibody comprising a sequence modification that inhibits presentation of the antibody by human leukocyte antigen (HLA) molecules.

In a suitable embodiment the anti-TNF-α antibody is infliximab or a fragment or variant thereof. In such an embodiment the modification is suitably one that inhibits presentation of the antibody by DRB1*03:01.

In a suitable embodiment the anti-TNF-α antibody is adalimumab or a fragment or variant thereof. In such an embodiment the modification is suitably one that inhibits presentation of the antibody by DQA1*05:05/DQB1*03:01.

Examples of particular modifications of antibodies, including modifications of infliximab and modifications of adalimumab, are discussed further in the detailed description of the invention.

In a second aspect, the invention provides a nucleic acid molecule comprising a nucleic acid sequence encoding an antibody of the invention. The antibody may be in accordance with any of the aspects or embodiments described herein.

In a third aspect the invention provides a pharmaceutical composition comprising an antibody of the invention and/or a nucleic acid molecule of the invention, and a pharmaceutical carrier. Suitably the pharmaceutical composition is an injectable composition.

The antibodies, nucleic acid molecules, and pharmaceutical compositions of the invention are all suitable for use in therapeutic applications.

Accordingly, in a fourth aspect, the invention provides a method of treating a disorder, the method comprising providing a therapeutically effective amount of an antibody, nucleic acid molecule or pharmaceutical composition of the invention to a subject in need thereof. Suitably the therapeutically effective amount of an antibody, nucleic acid molecule or pharmaceutical composition is administered by injection. Suitably the therapeutically effective amount of an antibody, nucleic acid molecule or pharmaceutical composition is administered by intravenous perfusion.

The fourth aspect also provides for the medical use of an antibody, nucleic acid molecule or pharmaceutical composition in accordance with the invention. Such methods of treatment or medical uses may be in the treatment of a disorder as considered elsewhere in this disclosure.

In a fifth aspect, there is provided a cell comprising a nucleic acid molecule in accordance with the second aspect of the invention.

In a sixth aspect, the invention provides a method of producing an antibody in accordance with the invention, the method comprising culturing a cell in accordance with the fifth aspect of the invention under conditions that permit the expression of an antibody of the invention.

### BRIEF DESCRIPTION OF THE DRAWING

FIG. 1A. Genetic polymorphism schematic with peptide binding motifs. The left panel shows representative chromosome six schematics of DRB1*03:01_DQA1*05:01_DQB1*02:01 and DRB1*11:01_DQA1*05:05_DQB1*03:01, respectively. The middle panel shows the resulting functional heterodimeric molecule from each DQ haplotype bound to a ligand, and the right panel shows the peptide binding motifs predicted for each haplotype. FIG. 1B-C Cox regression of individual alleles, split by drug treatment. B) HLA-DRB1*03:01, HLA-DQA1*05:01, and HLA-DQB1*02:01 have no association with adalimumab immunogenicity (HR=1.058, p=0.7978; HR=1.071, p=0.7555; and HR=1.061, p=0.7869, respectively), but do significantly associate to infliximab immunogenicity (HR=2.008, p=4.856836e-10; HR=1.928, p=3.071547e-09; and HR=1.926, p=2.919311e-09, respectively) C) Conversely, the alleles HLA-DRB1*11:01, HLA-DQA1*05:05, and HLA-DQB1*03:01 are all significantly associated with development of adalimumab immunogenicity (HR=2.912, p=2.87262e-05; HR=2.164, p=0.000108; and HR=1.489, p=0.031163, respectively), as well as, less significantly, infliximab (HR=1.377, p=0.056476, HR=1.406, p=0.004361; and HR=1.278, p=0.018322, respectively).

### DETAILED DESCRIPTION OF THE INVENTION

### Antibodies of the Invention Based Upon Infliximab

The inventors have found that DRB1*03:01 is particularly relevant to the presentation of sequences within infliximab that gives rise to the development of antibodies causing secondary immune-mediated loss of response to this drug. Accordingly, in a suitable embodiment, the anti-TNF-α antibody is infliximab or a fragment or variant thereof, and the modification is one that inhibits presentation of the antibody by DRB1*03:01.

In particular, the inventors have found that DRB1*03:01 interacts with and presents a motif within the sequence DILLTQSPAILSVSPGERVSF. This sequence (sequence set out as SEQ ID NO: 3) corresponds to residues 1 to 21 of the light chain of infliximab (as set out in SEQ ID NO: 1).

Accordingly, in a suitable embodiment, the modification that inhibits presentation of the antibody of the invention by DRB1*03:01 is a modification in the region corresponding to residues 1 to 21 of SEQ ID NO: 1 (sequence set out in SEQ ID NO: 3).

Suitably the modification that inhibits presentation of the antibody of the invention by DRB1*03:01 is a modification in the region corresponding to residues 1 to 20 of SEQ ID NO: 1 (sequence set out in SEQ ID NO: 4).

Suitably the modification that inhibits presentation of the antibody of the invention by DRB1*03:01 is a modification in the region corresponding to residues 1 to 18 of SEQ ID NO: 1 (sequence set out in SEQ ID NO: 5).

Suitably the modification that inhibits presentation of the antibody of the invention by DRB1*03:01 is a modification in the region corresponding to residues 1 to 16 of SEQ ID NO: 1 (sequence set out in SEQ ID NO: 6).

| | Corresponding | |
|---|---|---|
| | residues of | SEQ |
| Sequence | SEQ ID NO: 2 | ID NO: |
| DILLTQSPAILSVSPGERVSF | 1 to 21 | 3 |
| DILLTQSPAILSVSPGERVS | 1 to 20 | 4 |
| DILLTQSPAILSVSPGER | 1 to 18 | 5 |
| DILLTQSPAILSVSPG | 1 to 16 | 6 |

Suitably the modification is a modification of a residue corresponding to one or both of residues 10 and 11 of SEQ ID NO: 1. Suitably a residue corresponding to residue 10 of SEQ ID NO: 1 is modified. Suitably a residue corresponding to residue 11 of SEQ ID NO: 1 is modified. Suitably residues corresponding to both residues 10 and 11 of SEQ ID NO: 1 are modified.

In accordance with any of the embodiments disclosed above, when the modification is of a residue corresponding to residue 10 of SEQ ID NOS: 1, or 3-6, the modification is suitably a substitution with a glycine residue.

In accordance with any of the embodiments disclosed above, when the modification is of a residue corresponding to residue 11 of SEQ ID NOS: 1, or 3-6, the modification is suitably a substitution with a glycine residue.

In accordance with any of the embodiments disclosed above, when the modification is of residues corresponding to both residues 10 and 11 of SEQ ID NOS: 1, or 3-6, both modifications are suitably a substitution with a glycine residue.

An antibody in accordance with the invention may comprise the amino acid sequence set out in SEQ ID NO: 7 as a modification that inhibits presentation of the antibody by DRB1*03:01.

An antibody in accordance with the invention may comprise the amino acid sequence set out in SEQ ID NO: 8 as a modification that inhibits presentation of the antibody by DRB1*03:01.

An antibody in accordance with the invention may comprise the amino acid sequence set out in SEQ ID NO: 9 as a modification that inhibits presentation of the antibody by DRB1*03:01.

| Sequence | SEQ ID NO: |
|---|---|
| DILLTQSPAGLSVSPGERVSF | 7 |
| DILLTQSPAIGSVSPGERVSF | 8 |
| DILLTQSPAGGSVSPGERVSF | 9 |

In sequences listed in this specification (including those set out above, and those of SEQ ID NOs: 22 to 26, or 28 to 34, below) bold typeface indicates a modification as compared to the parent sequence. Such modifications represent subject matter that may be protected as a modification inhibiting presentation of an antibody of the invention by an HLA molecule.

The inventors have also found that DRB1*03:01 interacts with and presents a motif within the sequence KVQWKVDNALQSGNS. This sequence (SEQ ID NO: 10) corresponds to residues 145 to 159 of the light chain of infliximab (as set out in SEQ ID NO: 1).

Accordingly, in a suitable embodiment, the modification that inhibits presentation of the antibody of the invention by DRB1*03:01 is a modification in the region corresponding to residues 145 to 159 of SEQ ID NO: 1 (sequence set out in SEQ ID NO: 10).

Suitably the modification is a modification of a residue corresponding to residue 151 of SEQ ID NO: 1 (corresponding to residue 7 of SEQ ID NO: 10). Suitably the modification is a substitution with a glycine residue.

Accordingly, an antibody in accordance with the invention may comprise the amino acid sequence set out in SEQ ID NO: 11 as a modification that inhibits presentation of the antibody by DRB1*03:01.

(SEQ ID NO: 11)
KVQWKVGNALQSGNS.

For the purposes of the present invention, an antibody that is a “fragment” of infliximab may comprise at least 90% of the amino acid sequence set out in SEQ ID NO: 1 (save for any modification set out in SEQ ID NOS: 7-9 above). For example, a fragment of infliximab may comprise at least 91%, at least 92%, at least 93%, at least 94%, at least 95%, at least 96%, at least 97%, at least 98%, or at least 99% of the amino acid sequence set out in SEQ ID NO: 1 (save for any modification in accordance with the invention).

For the purposes of the present invention, an antibody that is a “variant” of infliximab may share at least 90% identity with the amino acid sequence set out in SEQ ID NO: 1 (save for any modification set out in SEQ ID NOS: 7-9 above). For example, a variant of infliximab may share at least 91%, at least 92%, at least 93%, at least 94%, at least 95%, at least 96%, at least 97%, at least 98%, or at least 99% identity with the amino acid sequence set out in SEQ ID NO: 1 (save for any modification in accordance with the invention).

### Antibodies of the Invention Based Upon Adalimumab

The inventors have found that DQA1*05:05/DQB1*03:01 is particularly relevant to the presentation of sequences within adalimumab that gives rise to the development of antibodies causing secondary immune-mediated loss of response to this drug. Accordingly, in a suitable embodiment, the anti-TNF-α antibody is adalimumab or a fragment or variant thereof, and the modification is one that inhibits presentation of the antibody by DQA1*05:05/DQB1*03:01.

In particular, the inventors have found that DQA1*05:05/DQB1*03:01 interacts with and presents a motif within the sequence EVQLVESGGGLVQPGRSLRLS. This sequence (sequence set out as SEQ ID NO: 15) corresponds to residues 1 to 21 of the light chain of adalimumab (as set out in SEQ ID NO: 12).

Accordingly, in a suitable embodiment, the modification that inhibits presentation of the antibody of the invention by DQA1*05:05/DQB1*03:01 is a modification in the region corresponding to residues 1 to 21 of SEQ ID NO: 12 (sequence set out in SEQ ID NO: 15).

In a suitable embodiment, the modification that inhibits presentation of the antibody of the invention by DQA1*05:05/DQB1*03:01 is a modification in the region corresponding to residues 1 to 20 of SEQ ID NO: 12 (sequence set out in SEQ ID NO: 16).

In a suitable embodiment, the modification that inhibits presentation of the antibody of the invention by DQA1*05:05/DQB1*03:01 is a modification in the region corresponding to residues 1 to 19 of SEQ ID NO: 12 (sequence set out in SEQ ID NO: 17).

In a suitable embodiment, the modification that inhibits presentation of the antibody of the invention by DQA1*05:05/DQB1*03:01 is a modification in the region corresponding to residues 1 to 18 of SEQ ID NO: 12 (sequence set out in SEQ ID NO: 18).

In a suitable embodiment, the modification that inhibits presentation of the antibody of the invention by DQA1*05:05/DQB1*03:01 is a modification in the region corresponding to residues 1 to 17 of SEQ ID NO: 12 (sequence set out in SEQ ID NO: 19).

In a suitable embodiment, the modification that inhibits presentation of the antibody of the invention by DQA1*05:05/DQB1*03:01 is a modification in the region corresponding to residues 1 to 16 of SEQ ID NO: 12 (sequence set out in SEQ ID NO: 20).

In a suitable embodiment, the modification that inhibits presentation of the antibody of the invention by DQA1*05:05/DQB1*03:01 is a modification in the region corresponding to residues 1 to 15 of SEQ ID NO: 12 (sequence set out in SEQ ID NO: 21).

| | Corresponding | |
|---|---|---|
| | residues of | SEQ |
| Sequence | SEQ ID NO: 12 | ID NO: |
| EVQLVESGGGLVQPGRSLRLS | 1 to 21 | 15 |
| EVQLVESGGGLVQPGRSLRL | 1 to 20 | 16 |
| EVQLVESGGGLVQPGRSLR | 1 to 19 | 17 |
| EVQLVESGGGLVQPGRSL | 1 to 18 | 18 |
| EVQLVESGGGLVQPGRS | 1 to 17 | 19 |
| EVQLVESGGGLVQPGR | 1 to 16 | 20 |
| EVQLVESGGGLVQPG | 1 to 15 | 21 |

Suitably the modification is a modification of a residue corresponding to one or more amino acid residues independently selected from the group consisting of: amino acids corresponding to residues 9; 10; 12; and 15 of SEQ ID NO: 12.

Suitably a residue corresponding to residue 9 of SEQ ID NO: 12 is modified. Suitably a residue corresponding to residue 10 of SEQ ID NO: 12 is modified. Suitably a residue corresponding to residue 12 of SEQ ID NO: 12 is modified. Suitably a residue corresponding to residue 15 of SEQ ID NO: 12 is modified.

Suitably two or more amino acids corresponding to residues independently selected from the group consisting of: amino acids residues 9; 10; 12; and 15 of SEQ ID NO: 12 are modified.

Suitably three or more amino acids corresponding to residues independently selected from the group consisting of: amino acids residues 9; 10; 12; and 15 of SEQ ID NO: 12 are modified.

Suitably amino acids corresponding all four amino acid residues 9; 10; 12; and 15 of SEQ ID NO: 12 are modified.

In accordance with any of the embodiments disclosed above, when the modification is of a residue corresponding to residue 9 of SEQ ID NOS: 12 or 15-21, the modification is suitably a substitution with a leucine residue.

In accordance with any of the embodiments disclosed above, when the modification is of a residue corresponding to residue 10 of SEQ ID NOS: 12 or 15-21, the modification is suitably a substitution with a valine residue.

In accordance with any of the embodiments disclosed above, when the modification is of a residue corresponding to residue 12 of SEQ ID NOS: 12 or 15-21, the modification is suitably a substitution with a leucine residue.

In accordance with any of the embodiments disclosed above, when the modification is of a residue corresponding to residue 15 of SEQ ID NOS: 12 or 15-21, the modification is suitably a substitution with a leucine residue.

An antibody in accordance with the invention may comprise the amino acid sequence set out in SEQ ID NO: 22 as a modification that inhibits presentation of the antibody by DQA1*05:05/DQB1*03:01.

An antibody in accordance with the invention may comprise the amino acid sequence set out in SEQ ID NO: 23 as a modification that inhibits presentation of the antibody by DQA1*05:05/DQB1*03:01.

An antibody in accordance with the invention may comprise the amino acid sequence set out in SEQ ID NO: 24 as a modification that inhibits presentation of the antibody by DQA1*05:05/DQB1*03:01.

An antibody in accordance with the invention may comprise the amino acid sequence set out in SEQ ID NO: 25 as a modification that inhibits presentation of the antibody by DQA1*05:05/DQB1*03:01.

An antibody in accordance with the invention may comprise the amino acid sequence set out in SEQ ID NO: 26 as a modification that inhibits presentation of the antibody by DQA1*05:05/DQB1*03:01.

| Sequence | SEQ ID NO: |
|---|---|
| EVQLVESGGVLVQPGRSLRLS | 22 |
| EVQLVESGLGLVQPGRSLRLS | 23 |
| EVQLVESGGGLLQPGRSLRLS | 24 |
| EVQLVESGGGLVQPLRSLRLS | 25 |
| EVQLVESGLVLLQPLRSLRLS | 26 |

The inventors have also found that DQA1*05:05/DQB1*03:01 interacts with and presents a motif within the sequence SKSTSGGTAALGCLV (SEQ ID NO: 27). This sequence corresponds to residues 136 to 150 of the heavy chain of adalimumab (as set out in SEQ ID NO: 13). Accordingly, a modification that inhibits presentation of the antibody by DQA1*05:05/DQB1*03:01 may be a modification of a residue corresponding to a residue in SEQ ID NO: 27.

Suitably the modification is a modification of a residue corresponding to one or more amino acid residues independently selected from the group consisting of: amino acids corresponding to residues 4; 5; 6; 7; 9; 10 and 12 of SEQ ID NO: 27 (respectively corresponding to residues 139; 140; 141; 142; 144; 145; and 147 of SEQ ID NO: 13)

In the event that the modification comprises a modification of a residue corresponding to residue 4 of SEQ ID NO: 27, the modification may be substitution with a leucine residue.

In the event that the modification comprises a modification of a residue corresponding to residue 5 of SEQ ID NO: 27, the modification may be substitution with a leucine residue.

In the event that the modification comprises a modification of a residue corresponding to residue 6 of SEQ ID NO: 27, the modification may be substitution with a leucine residue.

In the event that the modification comprises a modification of a residue corresponding to residue 7 of SEQ ID NO: 27, the modification may be substitution with a valine residue.

In the event that the modification comprises a modification of a residue corresponding to residue 9 of SEQ ID NO: 27, the modification may be substitution with a serine residue.

In the event that the modification comprises a modification of a residue corresponding to residue 10 of SEQ ID NO: 27, the modification may be substitution with a leucine residue.

In the event that the modification comprises a modification of a residue corresponding to residue 12 of SEQ ID NO: 27, the modification may be substitution with a leucine residue.

An antibody in accordance with the invention may comprise the amino acid sequence set out in SEQ ID NO: 28 as a modification that inhibits presentation of the antibody by DQA1*05:05/DQB1*03:01.

An antibody in accordance with the invention may comprise the amino acid sequence set out in SEQ ID NO: 29 as a modification that inhibits presentation of the antibody by DQA1*05:05/DQB1*03:01.

An antibody in accordance with the invention may comprise the amino acid sequence set out in SEQ ID NO: 30 as a modification that inhibits presentation of the antibody by DQA1*05:05/DQB1*03:01.

An antibody in accordance with the invention may comprise the amino acid sequence set out in SEQ ID NO: 31 as a modification that inhibits presentation of the antibody by DQA1*05:05/DQB1*03:01.

An antibody in accordance with the invention may comprise the amino acid sequence set out in SEQ ID NO: 32 as a modification that inhibits presentation of the antibody by DQA1*05:05/DQB1*03:01.

An antibody in accordance with the invention may comprise the amino acid sequence set out in SEQ ID NO: 33 as a modification that inhibits presentation of the antibody by DQA1*05:05/DQB1*03:01.

An antibody in accordance with the invention may comprise the amino acid sequence set out in SEQ ID NO: 34 as a modification that inhibits presentation of the antibody by DQA1*05:05/DQB1*03:01.

| Sequence | SEQ ID NO: |
|---|---|
| SKSTSGVTAALGCLV | 28 |
| SKSTSLVTAALGCLV | 29 |
| SKSTSLVTSALGCLV | 30 |
| SKSTSLVTSALLCLV | 31 |
| SKSTSLVTSLLLCLV | 32 |
| SKSLSLVTSLLLCLV | 33 |
| SKSLLLVTSLLLCLV | 34 |

### A Modified Antibody

For the purposes of the present disclosure, a “modification” may be considered to be any change to one or more amino acid residues that results in an amino acid sequence that does not correspond to the parent sequence (e.g. any of the sequences set out in SEQ ID NOS: 1, 2, 12 or 13).

Suitably the modification may comprise substitution of one or more amino acid residues within a recited sequence. Alternatively, the modification may comprise deletion of one or more amino acid residues within a recited sequence. A suitable modification may comprise addition of one or more amino acid residues within a recited sequence that are not present in the parent sequence.

Suitably an antibody in accordance with the present invention has properties that allow it to be used as a therapeutic antibody.

Suitably an antibody in accordance with the present invention is able to bind to, and inhibit the activity of, TNF-α. An antibody of the invention that is a fragment or derivative of a known therapeutic antibody (such as infliximab or adalimumab) may retain at least some of the parent antibody's capacity to bind to and inhibit the biological activity of TNF-α. Suitable means by which TNF-α activity, and hence the ability of an antibody to inhibit TNF-α, may be assessed will be well known to those skilled in the art. Examples of such suitable means include binding assays, by which a modified antibody's ability to bind to TNF-α, and thereby inhibit its activity, may be assessed.

### Presentation of Antibodies of the Invention by DRB1*03:01

As set out above, an antibody of the invention may comprise a modification that inhibits presentation of the antibody by DRB1*03:01. Presentation of such an antibody by DRB1*03:01 may be inhibited by at least 10%, at least 20%, least 30%, at least 40%, least 50%, at least 60%, least 70%, at least 80%, least 90%, or even by 100% as compared to the presentation of the parent (or other control unmodified antibody) by DRB1*03:01.

The skilled person will be aware of suitable assays by which presentation of antibodies by DRB1*03:01 may be assessed, including, but not limited to, those set out below.

### Presentation of Antibodies of the Invention by DQA1*05:05/DQB1*03:01

As set out above, an antibody of the invention may comprise a modification that inhibits presentation of the antibody by DQA1*05:05/DQB1*03:01. Presentation of such an antibody by DQA1*05:05/DQB1*03:01 may be inhibited by at least 10%, at least 20%, least 30%, at least 40%, least 50%, at least 60%, least 70%, at least 80%, least 90%, or even by 100% as compared to the presentation of the parent (or other control unmodified antibody) by DQA1*05:05/DQB1*03:01.

The skilled person will be aware of suitable assays by which presentation of antibodies by DQA1*05:05/DQB1*03:01 may be assessed, including, but not limited to, those set out below.

### Assays for Assessment of Presentation of Antibodies of the Invention

Monocyte-derived dendritic cells that express molecule presentation of antibodies by which is to be assessed, or cells that are capable of up-taking antigen (i.e. monocyte cell lines that can be differentiated into macrophages) that express such molecule, may be used in suitable assays. Antibodies of the invention, or fragments of such antibodies, may be added to the cells, and subsequent analysis of uptake and presentation of the antibody can then be assessed using immunopeptidomics (the sequencing of HLA-associated peptides using liquid chromatography mass spectrometry).

It will be appreciated that the assays set out above may be used to assess presentation of antibodies by DRB1*03:01 or by DQA1*05:05/DQB1*03:01 as required, by selection of cells expressing the requisite presentation molecule.

In addition, or as an alternative, to the methods described above, binding can further be assessed by the production of tetramers, and use of refolding assays.

### Disorders to be Treated

The fourth aspect of the invention provides methods of treatment and medical uses of the antibodies, nucleic acid molecules and pharmaceutical compositions of the invention. The disorder to be treated may be one associated with pathological activity of TNF-α. The disorder may be an autoimmune disease. Suitably the disorder may be selected from the group consisting of: Crohn's disease; ulcerative colitis; rheumatoid arthritis; psoriatic arthritis; psoriasis; ankylosing spondylitis; and Behçet's disease.

A method of treatment, the method comprising providing a therapeutically effective amount of an agent selected from the group consisting of:
an antibody according to the first aspect of the invention;
a nucleic acid molecule according to the second aspect of the invention; and
a pharmaceutical composition according to the third aspect of the invention; to a subject in need of such treatment.

### Cells of the Invention, and Methods of Producing the Antibodies of the Invention

The fifth aspect of the invention provides cells comprising a nucleic acid molecule in accordance with the second aspect of the invention. In a suitable embodiment, the cells are selected for their suitability to express therapeutically effective antibodies. The cells of the invention provide suitable means by which antibodies of the invention may be produced.

Thus, the sixth aspect of the invention provides a method of producing an antibody in accordance with the invention, the method comprising culturing a cell in accordance with the fifth aspect of the invention under conditions that permit the expression of an antibody of the invention.

### EXAMPLES

### Introduction

The global occurrence of inflammatory bowel diseases (IBD), including Crohn's disease (CD), is rising, with prevalence of 0.5-1.0% reported in Europe and North America[1]. Biological therapies are a mainstay of patient management, with monoclonal antibodies against Tumour Necrosis Factor (TNF), infliximab and adalimumab, widely used as first-line agents. Highly effective in some individuals, continued use of these drugs is limited by primary non-response (10-40%) or secondary immune-mediated loss of response (24-46%) to treatment in the first year[2].

HLA class II polymorphisms have been implicated as contributory to disease progression in numerous autoimmune diseases. A recent study based upon data from the UK nation-wide analysis of anti-TNF response in CD implicates HLA-DQA1*05 in the development of antibodies against both anti-TNF biologics, with important clinical implications[3]. In this study, HLA-DQA1*05 represents a group of allelic variants, each encoding a distinct protein: HLA-DQA1*05:01, HLA-DQA1*05:03 (A182S) and HLA-DQA1*05:05 (A11T, signal peptide).

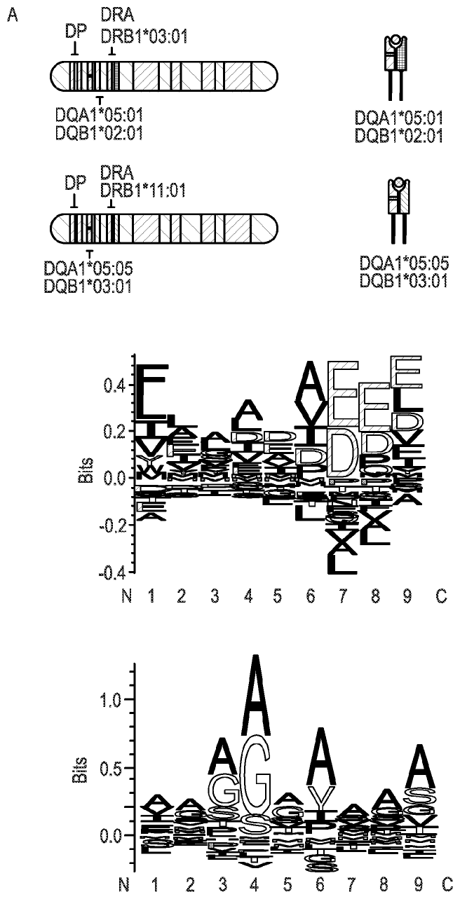

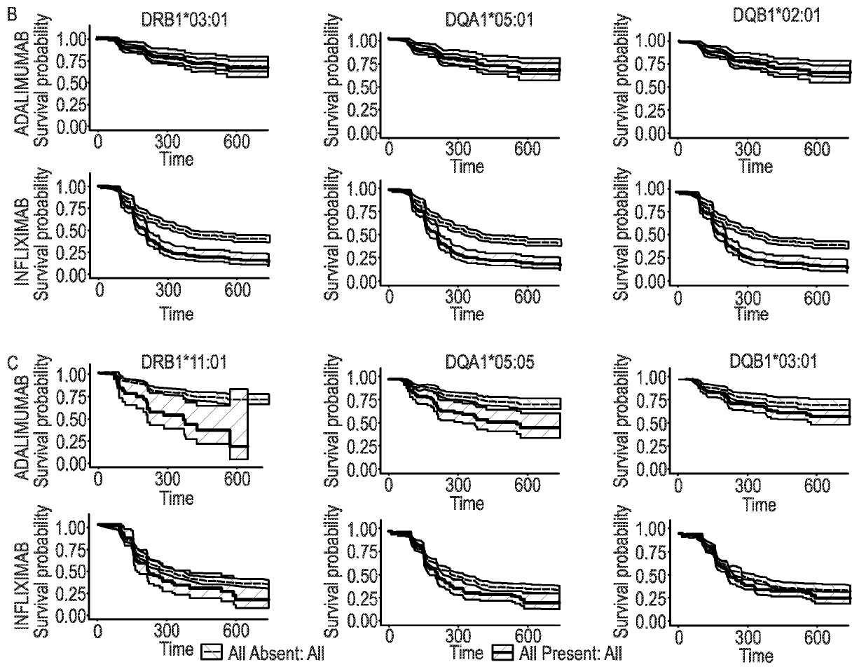

The HLA class II gene locus that includes HLA-DQA1 gene is a complex locus, encoding functional and highly polymorphic molecules that present peptide antigens to T helper cells which provide stimulus to B cell maturation and antibody formation. HLA class II molecules are heterodimers consisting of an alpha (A loci) and beta chain (B loci). The combination of the alpha chain with a particular beta chain, as well as amino acid differences within and outside the peptide binding groove, can affect trafficking and ligand binding (FIG. 1A). It is further known that within this region a high level of linkage disequilibrium results in the frequent occurrence of extended haplotypes[4], directly linking multiple HLA allele variants across the three major class II genes DR, DQ and DP[5].

With this biological context in mind, we hypothesized that, due to the different molecular composition of adalimumab and infliximab, their antigenic presentation and resulting potential to induce immunogenicity would differ, and be directly dependent not only on the alpha chain, but also the pairing beta chain, which mostly directs antigen binding specificity.

### Methods

Using the PANTS discovery cohort [3], 1240 biologic-naïve patients with CD starting either infliximab or adalimumab, we explored the cohort at 4D resolution, running pairwise Pearson correlations between each HLA-DQA1*05 variant and extended haplotypes in order to determine whether they should be considered as individual factors. We split the original data based on treatment, and applied Cox regression to each cohort to examine the significance of each HLA variant to immunogenicity conditioned on treatment method.

Immunogenicity is defined as an anti-drug antibody concentration of ≥10 AU/mL, irrespective of drug level, at any time point across the initial 12 month study and two year follow-up. Anti-drug antibodies were measured in all serum samples, at every visit, for every patient[3].

### Results

Pearson correlation analysis indicates weak correlation between the three HLA-DQA1*05 protein variants (HLA-DQA1*05:01 vs HLA-DQA1*05:03 ρ=−0.0219; HLA-DQA1*05:01 vs HLA-DQA1*05:05 ρ=−0.0921; HLA-DQA1*05:03 vs HLA-DQA1*05:05 ρ=−0.0196), indicating these variants are biologically unrelated and should be considered separately. Analysis on HLA-DQA1*05:03 was not progressed, as it exists in only one individual in the PANTS cohort.

Conversely, a strong correlation is evident between alleles HLA-DQA1*05:01, HLA-DQB1*02:01, and HLA-DRB1*03:01 (HLA-DQA1*05:01 vs HLA-DQB1*02:01 ρ−1; HLA-DQA1*05:01 vs HLA-DRB1*03:01 ρ=0.9976), indicating HLA alpha chain HLA-DQA1*05:01 pairs exclusively with HLA beta chain HLA-DQB1*02:01, and almost exclusively with HLA-DRB1*03:01, confirming the known linkage disequilibrium across this region in this cohort[5].

HLA-DQA1*05:05 is also strongly correlated with HLA-DQB1*03:01 and HLA-DRB1*11:01 (HLA-DQA1*05:05 vs HLA-DQB1*03:01 ρ=0.6782; HLA-DQA1*05:05 vs HLA-DRB1*11:01 ρ=0.6481), noting that HLA-DQB1*03:01 occurs in combination with alpha chains other than HLA-DQA1*05:05 but not vice versa.

Cox regression in the drug-specific cohorts demonstrates alleles HLA-DQA1*05:01, HLA-DQB1*02:01, and HLA-DRB1*03:01 have no association with adalimumab immunogenicity (HR=1.071, p=0.7555; HR=1.061, p=0.7869; and HR=1.058, p=0.7978, respectively). Conversely, alleles HLA-DQA1*05:05, HLA-DQB1*03:01, and HLA-DRB1*11:01 are all significantly associated with development of adalimumab immunogenicity (HR=2.164, p=0.000108; HR=1.489, p=0.031163; and HR=2.912, p=2.87262e-05, respectively).

Alternatively, HLA-DQA1*05:01, HLA-DQB1*02:01, and HLA-DRB1*03:01 demonstrate highly significant association to infliximab immunogenicity (HR=1.928, p=3.071547e-09; HR=1.926, p=2.919311e-09; and HR=2.008, p=4.856836e-10, respectively), while HLA-DQA1*05:05, HLA-DQB1*03:01, and HLA-DRB1*11:01 show some significance, but the association is reduced (HR=1.406, p=0.004361; HR=1.278, p=0.018322; and HR=1.377, p=0.056476, respectively).

Finally, we observe additional associations of immunogenicity for each drug in the variant of the HLA-DRB1 gene: HLA-DRB1*03:01 (infliximab, HR=2.008, p=4.856836e-10) and HLA-DRB1*11:01 (adalimumab, HR=2.912, p=2.87262e-05).

### Discussion

Our analysis demonstrates that grouping together different HLA-DQA1*05 alleles has important limitations that critically impact the interpretation of these data.

Splitting individual alleles and extended haplotypes by treatment option (FIG. 1), we show a clear difference between their effects. HLA-DQA1*05:01 and its extended haplotype are significantly associated with immunogenicity against infliximab but NOT adalimumab (FIG. 1B), while HLA-DQA1*05:05 and its extended haplotype are significantly associated with immunogenicity against adalimumab AND, less strongly, infliximab (FIG. 1C).

This distinction is important in the context of potential clinical translation. These data demonstrate no evidence for the development of adalimumab immunogenicity for patients who possess only the HLA-DQA1*05:01 variant and extended haplotype. Therefore, if one relies on 2D resolution of the HLA-DQA1*05supertype, 144 individuals in the study cohort (11.6%) currently receiving adalimumab with the HLA-DQA1*05:01 extended haplotype would be mischaracterised as at risk of immunogenicity.

Finally, our analysis shows HLA-DQA1*05:01, HLA-DQB1*02:01, and HLA-DRB1*03:01 exhibit 100% linkage disequilibrium, making it impossible to determine causality based upon Cox regression analysis (FIG. 1B)[5-7]. Moreover, the alleles HLA-DQA1*05:05, HLA-DQB1*03:01, and HLA-DRB1*11:01 are similarly indistinguishable (FIG. 1C).

These data highlight the complexities of deriving biological and clinical understanding of the mechanisms underlying immunogenicity to anti-TNF therapy from genetic studies of the HLA region. The effects of linkage disequilibrium and the heterodimeric nature of the antigen presenting molecules are all important considerations. In the context of class II molecular structure and function, the contributions of both the alpha and beta chains must be considered (FIG. 1A). Furthermore, the heterogeneity of response to either drug suggest potentially distinct underlying mechanisms of immunogenicity.

We conclude association studies within the HLA gene locus require single HLA variant analysis to allow accurate interpretation of the data for biological understanding and potential clinical application.

### REFERENCES

1. Steenholdt, C., et al., Optimizing Treatment with TNF Inhibitors in Inflammatory Bowel Disease by Monitoring Drug Levels and Antidrug Antibodies. Inflamm Bowel Dis, 2016. 22(8): p. 1999-2015.
2. Kennedy, N. A., et al., Predictors of anti-TNF treatment failure in anti-TNF-naive patients with active luminal Crohn's disease: a prospective, multicentre, cohort study. Lancet Gastroenterol Hepatol, 2019. 4(5): p. 341-353.
3. Sazonovs, A., et al., HLA-DQA1*05 Carriage Associated With Development of Anti-Drug Antibodies to Infliximab and Adalimumab in Patients With Crohn's Disease. Gastroenterology, 2020. 158(1): p. 189-199.
4. de Bakker, P. I., et al., A high-resolution HLA and SNP haplotype map for disease association studies in the extended human MHC. Nat Genet, 2006. 38(10): p. 1166-72.
5. Price, P., et al., The genetic basis for the association of the 8.1 ancestral haplotype (A1, B8, DR3) with multiple immunopathological diseases. Immunol Rev, 1999. 167: p. 257-74.
6. Brzyski, D., et al., Controlling the Rate of GWAS False Discoveries. Genetics, 2017. 205(1): p. 61-75.
7. Lee, J. C., et al., Genome-wide association study identifies distinct genetic contributions to prognosis and susceptibility in Crohn's disease. Nat Genet, 2017. 49(2): p. 262-268.

SEQUENCE INFORMATION
Infliximab Sequence
SEQ ID NO: 1 Light Chain of infliximab
DILLTQSPAILSVSPGERVSFSCRASQFVGSSIHWYQQRTNGSPRLL
IKYASESMSGIPSRFSGSGSGTDFTLSINTVESEDIADYYCQQSHSW
PFTFGSGTNLEVKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
SEQ ID NO: 2 Heavy Chain of infliximab
EVKLEESGGGLVQPGGSMKLSCVASGFIFSNHWMNWVRQSPEKGLEW
VAEIRSKSINSATHYAESVKGRFTISRDDSKSAVYLQMTDLRTEDTG
VYYCSRNYYGSTYDYWGQGTTLTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKT
Adalimumab Sequence
SEQ ID NO: 12 Light Chain: of adalimumab
DIQMTQSPSSLSASVGDRVTITCRASQGIRNYLAWYQQKPGKAPKLL
IYAASTLQSGVPSRFSGSGSGTDFTLTISSLQPEDVATYYCQRYNRA
PYTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGE
SEQ ID NO: 13 Heavy Chain: of adalimumab
EVQLVESGGGLVQPGRSLRLSCAASGFTFDDYAMHWVRQAPGKGLEW
VSAITWNSGHIDYADSVEGRFTISRDNAKNSLYLDMNSLRAEDTAVY
YCAKVSYLSTASSLDYWGQGTLVTVSSASTKGPSVFPLAPSSKSTSG
GTAALGCLVKDYFPEPVTVSWNSGALTSGVHTFPAVLQSSGLYSLSS
VVTVPSSSLGTQTYICNVNHKPSNTKVDKKI

In sequences listed in this specification, bold typeface indicates a modification as compared to the parent sequence. Such modifications represent subject matter that may be protected as a modification inhibiting presentation of an antibody of the invention by an HLA molecule.

1. An anti-TNF-α antibody comprising a sequence modification that inhibits presentation of the antibody by human leukocyte antigen (HLA) molecules.

2. An antibody according to claim 1, wherein the anti-TNF-α antibody is infliximab or a fragment or variant thereof.

3. An antibody according to claim 1 or claim 2, wherein the modification is suitably one that inhibits presentation of the antibody by DRB1*03:01.

4. An antibody according to any preceding claim, wherein the modification that inhibits presentation of the antibody of the invention by DRB1*03:01 is a modification in the region corresponding to residues 1 to 21 of SEQ ID NO: 1

5. An antibody according to any preceding claim, wherein the modification is a modification of a residue corresponding to one or both of residues 10 and 11 of SEQ ID NO: 1.

6. An antibody according to claim 5, wherein the modification is of a residue corresponding to residue 10 of SEQ ID NO: 1 and the modification is a substitution with a glycine residue.

7. An antibody according to claim 5 or claim 6, wherein the modification is of a residue corresponding to residue 11 of SEQ ID NO: 1 and the modification is a substitution with a glycine residue.

8. An antibody according to claim 6 or claim 7, wherein the modification is of residues corresponding to both residues 10 and 11 of SEQ ID NO: 1, and both modifications are a substitution with a glycine residue.

9. An antibody according to any of claims 1 to 3, wherein the modification that inhibits presentation of the antibody of the invention by DRB1*03:01 is a modification in the region corresponding to residues 145 to 159 of SEQ ID NO: 1

10. An antibody according to claim 1, wherein the anti-TNF-α antibody is adalimumab or a fragment or variant thereof.

11. An antibody according to claim 1, wherein the modification is one that inhibits presentation of the antibody by DQA1*05:05/DQB1*03:01.

12. A nucleic acid molecule comprising a nucleic acid sequence encoding an antibody of any of claims 1 to 11.

13. An antibody according to any of claims 1 to 11, for use as a medicament.

14. An antibody for use according to claim 13, in the treatment of a disorder associated with pathological activity of TNF-α.

15. An antibody for use according to claim 13 or claim 14, in the treatment of an autoimmune disease.

16. An antibody for use according to any of claims 13 to 15, in the treatment of a disorder selected from the group consisting of: Crohn's disease; ulcerative colitis; rheumatoid arthritis; psoriatic arthritis; psoriasis; ankylosing spondylitis; and Behçet's disease

17. A pharmaceutical composition comprising an antibody of any of claims 1 to 11 and/or a nucleic acid molecule of claim 12, and a pharmaceutical carrier.

18. A pharmaceutical composition according to claim 17, wherein the pharmaceutical composition is an injectable composition.

19. A pharmaceutical composition according to claim 17 or claim 18, for use in the treatment of a disorder associated with pathological activity of TNF-α.

20. A pharmaceutical composition for use according to claim 19, in the treatment of an autoimmune disease.

21. A pharmaceutical composition for use according to claim 19 or claim 20, in the treatment of a disorder selected from the group consisting of: Crohn's disease; ulcerative colitis; rheumatoid arthritis; psoriatic arthritis; psoriasis; ankylosing spondylitis; and Behçet's disease.

22. A method of treatment, the method comprising providing a therapeutically effective amount of an agent selected from the group consisting of:
an antibody according to any of claims 1 to 11;
a nucleic acid molecule according to claim 12; and
a pharmaceutical composition according to claims 17 or claim 18;
to a subject in need of such treatment.

23. A method of treatment according to claim 22, for treatment of a disorder associated with pathological activity of TNF-α.

24. A method of treatment according to claim 22 or claim 23, for treatment of an autoimmune disease.

25. A method of treatment according to any of claims 22 to 24, for treatment of a disorder selected from the group consisting of: Crohn's disease; ulcerative colitis; rheumatoid arthritis;
psoriatic arthritis; psoriasis; ankylosing spondylitis; and Behçet's disease.
